(19) United States
(12) Patent Application Publication
(10) Pub. No.: US 2021/0077825 A1
(43) Pub. Date: Mar. 18, 2021

(54) SKIN CARE APPARATUS USING LIGHT

(71) Applicant: Samsung Electronics Co., Ltd., Suwon-si (KR)

(72) Inventors: Gwangjin JUNG, Suwon-si (KR); Byoungwoo KO, Suwon-si (KR); Jungkwon KIM, Suwon-si (KR); Chunseong KIM, Suwon-si (KR)

(21) Appl. No.: 17/023,236

(22) Filed: Sep. 16, 2020

(30) Foreign Application Priority Data
Sep. 18, 2019 (KR) 10-2019-0114828

(51) Int. Cl. A61N 5/06

(52) U.S. Cl. CPC A61N 5/0616; A61N 2005/0632; A61N 2005/0626; A61N 2005/063; A61N 2005/0647

(57) ABSTRACT

Disclosed is a skin care apparatus using light. The skin care apparatus includes a plurality of light sources, a mask that is provided with the plurality of light sources and formed with a plurality of light passing holes through which light emitted from the plurality of light sources passes. The skin care apparatus also includes a light blocking rib formed in at least one light passing hole among the plurality of light passing holes.

### CROSS-REFERENCE TO RELATED APPLICATION

This application is based on and claims priority under 35 U.S.C. § 119 to Korean Patent Application No. 10-2019-0114828 filed on Sep. 18, 2019 in the Korean Intellectual Property Office, the disclosure of which is incorporated by reference herein in its entirety.

### BACKGROUND

### 1. Field

The disclosure relates to a skin care apparatus using light and, more particularly, to a skin care apparatus for promoting biochemical reaction in a skin by irradiating a face of a human body with light.

### 2. Description of Related Art

Given that anti-aging, meaning stopping an aging process, becomes a trend, not only a cosmetic industry but also an industry of manufacturing a skin care apparatus has been developing and selling products associated with anti-aging.

Recently, a skin care apparatus to promote biochemical reaction in the skin by irradiating skin with light emitted from a light emitting diode (LED) has been released.

In the related art, when the skin is irradiated with light using an optical skin care device, energy is supplied even to a dermal layer which determine the elasticity of the skin, thereby helping blood circulation and obtaining an effect of improving skin such as alleviating freckles, wrinkles, erythema, and enhancing whitening of the skin.

The related-art optical skin care device is composed of a mask that is mountable on a face of a human body, an LED module mounted on the mask to irradiate a face of a user wearing a mask with light, and an eye-shield for preventing glare.

However, in the related-art optimal skin care device, if a user wears a mask on the face, an eye-shield for preventing glare may press eyes or an area around the eyes, thus causing inconvenience or even pain to a user using the mask.

### SUMMARY

Provided is a skin care apparatus minimizing glare caused by light while not pressing the eye or an area around the eye of a user wearing a mask.

According to an embodiment, a skin care apparatus includes a plurality of light sources, a mask which is provided with the plurality of light sources and formed with a plurality of light passing holes through which light emitted from the plurality of light sources passes, and a light blocking rib formed in at least one light passing hole among the plurality of light passing holes.

The light blocking rib may be formed at a light passing hole that is most adjacent to an eye of a person wearing a mask.

The light blocking rib may protrude in a first direction toward an outer side of the mask, or protrude in a second direction that is opposite to the first direction.

The mask may be formed with a viewing hole to expose an eye when the mask is worn on a face.

The light blocking rib may be formed at a light passing hole positioned most adjacently to the viewing hole.

The light blocking ribs may be formed in plural, the plurality of the light blocking ribs being formed in each of a predetermined number of light passing holes in a direction distancing from a light passing hole positioned most adjacently to the viewing hole.

The plurality of light blocking ribs may be positioned at a right angle with respect to an inner surface of the mask.

The plurality of light blocking ribs may be positioned to be inclined with respect to the inner surface of the mask.

The plurality of light blocking ribs may be positioned such that an angle with respect to the inner surface of the mask increases or decreases as the light blocking ribs gradually distance from the viewing hole.

The plurality of light blocking ribs may be positioned at different angles with respect to the inner surface of the mask.

The plurality of light blocking ribs may be formed to have a shorter length as the light blocking ribs gradually distance from the viewing hole.

Each of the plurality of light blocking holes may be positioned along a length direction of the viewing hole and is positioned at intervals in a direction distancing from the viewing hole, and a plurality of light sources may be positioned at each light passing hole.

At least one of the plurality of light sources may be positioned in each of the plurality of light passing holes.

The viewing holes may be provided in a pair to correspond to a left eye and a right eye, respectively.

The skin care apparatus may further include at least one additional light passing hole positioned between the pair of viewing holes, and a pair of light blocking ribs that are adjacently positioned to the pair of viewing holes may be formed in the at least one additional light passing hole.

The skin care apparatus may further include at least one positioned additional light passing hole at both ends of the pair of viewing holes, and an additional light blocking rib may be formed in a side adjacent to each additional light passing hole.

The skin care apparatus may further include at least one positioned additional light passing hole at both ends which are positioned farthest from the viewing hole, and a light blocking rib may be formed in each additional light passing hole in a side adjacent to the viewing hole, respectively.

The light passing hole may be formed to be a circular shape, an oval shape, or a polygonal shape, and the light blocking rib may be disposed at a side adjacent to the viewing hole.

Provided is a skin care apparatus including a mask including an outer case, an inner case coupled with the outer case, and a viewing hole formed to penetrate the outer case and the inner case, a plurality of light sources positioned in the outer case, a plurality of light passing holes formed in the inner case through which light emitted from the plurality of light sources passes, and a plurality of light blocking ribs, positioned in edges of each of the plurality of light passing holes, protruding in a first direction toward the outer case or protruding in a second direction opposite to the first direction.

The plurality of light blocking ribs may be formed to be right-angled or inclined with respect to the inner case, respectively.

The inner case may be made of an opaque material.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a diagram of a state of wearing a skin care apparatus on a face according to an embodiment;

FIG. 2 illustrates a partial sectional diagram of a skin care apparatus according to an embodiment;

FIG. 3 illustrates a rear view of a skin care apparatus according to an embodiment;

FIG. 4 illustrates a partial sectional diagram of various arrangement examples of a light blocking rib provided in a skin care apparatus according to an embodiment;

FIG. 5 illustrates a partial sectional diagram of various arrangement examples of a light blocking rib provided in a skin care apparatus according to an embodiment;

FIG. 6 illustrates a partial sectional diagram of various arrangement examples of a light blocking rib provided in a skin care apparatus according to an embodiment;

FIG. 7 illustrates a partial sectional diagram of various arrangement examples of a light blocking rib provided in a skin care apparatus according to an embodiment;

FIG. 8 illustrates a partial sectional diagram of various arrangement examples of a light blocking rib provided in a skin care apparatus according to an embodiment;

FIG. 9 illustrates a schematic diagram of various arrangements of an LED module and a light passing hole formed on the skin care apparatus according to an embodiment;

FIG. 10 illustrates a schematic diagram of various arrangements of an LED module and a light passing hole formed on the skin care apparatus according to an embodiment;

FIG. 11 illustrates a schematic diagram of various arrangements of an LED module and a light passing hole formed on the skin care apparatus according to an embodiment;

FIG. 12A illustrates a schematic diagram of various shapes of a light passing hole formed at a skin care apparatus according to an embodiment;

FIG. 12B illustrates a schematic diagram of various shapes of a light passing hole formed at a skin care apparatus according to an embodiment;

FIG. 12C illustrates a schematic diagram of various shapes of a light passing hole formed at a skin care apparatus according to an embodiment;

FIG. 13 illustrates a schematic diagram of an arrangement of a light blocking rib formed in each light passing hole and the light passing hole positioned around the viewing hole of the skin care apparatus according to an embodiment;

FIG. 14 illustrates a schematic diagram of an arrangement of a light blocking rib formed in each light passing hole and the light passing hole positioned around the viewing hole of the skin care apparatus according to an embodiment;

FIG. 15 illustrates an exploded perspective view of a light blocking rib provided in the skin care apparatus being formed separately from an inner case according to an embodiment; and

FIG. 16 illustrates a block diagram of a skin care apparatus according to an embodiment.

### DETAILED DESCRIPTION

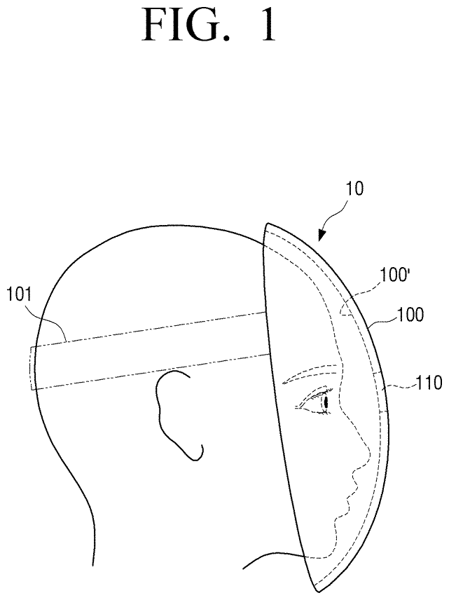

FIGS. 1 through 16, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

In describing the disclosure, detailed descriptions of the related known functions or configurations are omitted when it is determined that the specific description thereof may obscure the gist of the disclosure. In addition, the following examples may be modified in various other forms, and the scope of the technical concepts of the disclosure is not limited to the examples below. Rather, these embodiments are provided so that this disclosure will be thorough and complete, and will fully convey the scope of the disclosure to those skilled in the art.

Hereinafter, embodiments of the disclosure will be described with reference to the accompanying drawings. However, this disclosure is not intended to limit the embodiments described herein but includes various modifications, equivalents, and/or alternatives. In the context of the description of the drawings, like reference numerals may be used for similar components.

In the description, the terms “first,” “second,” and so forth are used to describe diverse elements regardless of their order and/or importance, and to discriminate one element from other elements, but are not limited to the corresponding elements.

A singular expression includes a plural expression, unless otherwise specified. It is to be understood that the terms such as “comprise” or “include” are used herein to designate a presence of a characteristic, number, step, operation, element, component, or a combination thereof, and not to preclude a presence or a possibility of adding one or more of other characteristics, numbers, steps, operations, elements, components or a combination thereof.

Hereinafter, a skin care apparatus according to an embodiment will be described with reference to the accompanying drawings.

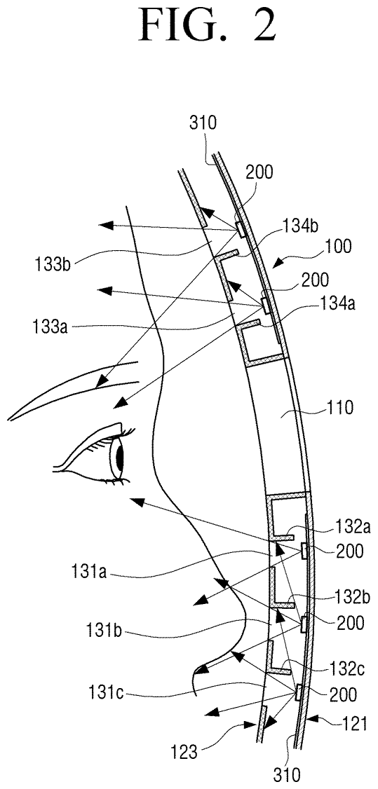

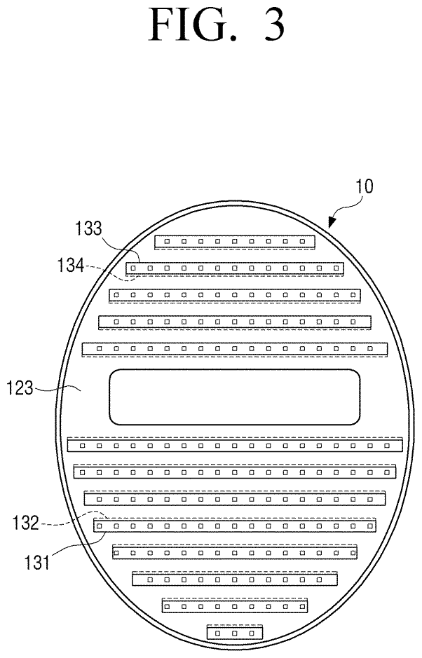

FIG. 1 illustrates a diagram of a state of wearing a skin care apparatus on a face according to an embodiment, FIG. 2 illustrates a partial sectional diagram of a skin care apparatus according to an embodiment, and FIG. 3 illustrates a rear view of a skin care apparatus according to an embodiment.

Referring to FIG. 1, a skin care apparatus 10 according to an embodiment can include a mask 100 that is sized to cover the entire face so as to irradiate the entire face with light (e.g., visible rays or near infrared rays).

The skin care apparatus 10 may include a fixing band 101 for wearing the mask 100 on the face. The fixing band 101 is made of a material having elasticity so that the mask 100 worn on the face is not movable. The skin care apparatus 10 may include a member of a cap shape that can be worn on the head or a member of an eyeglass frame shape that can be worn on the ear, as an alternative of the fixing band 101.

The mask 100, when worn on the face, may be positioned at a predetermined interval such that an inner surface 100′ of the mask 100 is not in contact with the face. For this purpose, a predetermined spacer (not shown) in contact with a portion of the face may be positioned on the inner surface 100′ of the mask. The spacer may be integrally formed with the mask 100 or may be formed as a separate member. Here, the inner surface 100′ of the mask 100 may be the same surface as a first surface (a surface facing the face when worn on the face) of an inner case 123 (see FIG. 2), which will be described later.

The mask 100, when worn on the face, may be provided with a viewing hole 110 at a position corresponding to the eye of the user. Specifically, the viewing hole 110 is formed to penetrate an outer case 121 and an inner case 123, which will be described later. The viewing hole 110 provided in the mask 100 can be formed in a transverse direction of the mask 100, and can be formed to have a length such that the left eye and the right eye can be exposed to the outside of the mask 100 (see FIG. 3).

Referring to FIGS. 2 and 3, a structure of the mask 100 will be described in greater detail.

Referring to FIG. 2, the mask 100 may be formed of the outer case 121 and the inner case 123 coupled with the outer case 121.

Hereinafter, a first surface of the outer case 121 is defined as a surface corresponding to the inner case 123, and a second surface of the outer case 121 is defined as a surface facing the outside of the mask 100. In addition, the first surface of the inner case 123 is defined as a surface facing the face when the mask 100 is worn, and the second surface of the inner case 123 is defined as a surface corresponding to the first surface of the outer case 121.

The outer case 121 forms an appearance of the mask 100 and a plurality of light sources 200 may be positioned on the first surface of the outer case 121 to which the inner case 123 is coupled.

Each light source 200 positioned on the first surface of the outer case 121 may be an LED module having an LED element outputting a wavelength of a visible light region or an LED element outputting a wavelength of a near infrared region. However, the light source is not limited to the LED module described above, and is applicable to any light source which is not harmful to the human body when being irradiated to the skin and can obtain the effect of improving the skin. Hereinafter, the “light source” is limited to “LED module” for convenience, and the light source and the LED module may use the same reference numeral.

The outer case 121 may be provided with a driver 300 for driving the plurality of LED modules 200 in the first surface. The driver 300 may include a printed circuit board 310 mounted with a driving integrated circuit (IC, not shown) for the LED module.

The printed circuit board 310 may be mounted with a plurality of LED modules 200. The printed circuit board 310 may be separably fixed to the first surface of the outer case 121 through a fastening member, such as a fastening screw or a well-known snap fit structure. The printed circuit board 310 may be formed of a printed circuit board with a hard material that has a predetermined strength and thus is not bendable or a flexible printed circuit board (FPCB) having flexibility.

The outer case 121 may have a first surface formed in a plane or a curved surface. When the first surface of the outer case 121 is formed of a curved surface, the printed circuit board 310 can be arranged to bend at a predetermined curvature along the first surface of the outer case 121 by applying the FPCB. In this case, since the printed circuit board 310 is deformable to correspond to the first surface of the outer case 121 and thus may contribute to form a thin thickness of the mask 100.

The inner case 123 may have a plurality of light passing holes 131, 133. The plurality of light passing holes 131, 133 may be formed at a position corresponding to the LED module 200 so that light emitted from each LED module 200 may pass therethrough.

The inner case 123 may be coupled to the outer case 121 at a predetermined interval from the outer case 121. Accordingly, a predetermined space can be present between the outer case 121 and the inner case 123. The space may be formed at an interval so that the plurality of LED modules 200 positioned on the first surface of the outer case 121 are not in contact with the second surface of the inner case 123.

The inner case 123 may be made of an opaque material through which light may not pass. This allows light emitted from the plurality of LED modules 200 to be irradiated through the plurality of light passing holes 131 and 133 to the skin of the face.

The inner case 123 may be formed to have a curvature corresponding to a curvature of the outer case 121. In this case, the inner case 123 may be formed to be convex or concave in the same direction as the outer case 121.

Referring to FIG. 2, each of the light passing holes 131 and 133 may have, for example, a band shape along the transverse direction of the mask 100. The plurality of light passing holes 131 and 133 may be positioned with a predetermined interval in a downward direction and an upward direction of the mask 100 with respect to the viewing hole 110.

In the case where the first surface of the inner case 123 is formed of a curved surface having a predetermined curvature, the plurality of light passing holes 131 and 133 are formed along the first surface of the inner case 123, and thus can be formed to have the same curvature as the first surface of the inner case 123.

Referring to FIG. 3, a plurality of light blocking ribs 132, 134 to prevent glare by blocking irradiation of light emitted from the LED module 200 toward the user's eyes may be formed in the plurality of light passing holes 131, 133.

The light blocking ribs 132, 134 may be formed at some of the plurality of light passing holes 131, 133 (see FIG. 2) or may be formed at all of the plurality of light passing holes 131, 133 (see FIG. 3).

When the light blocking rib is formed on only a part of the plurality of light passing holes, the light blocking ribs 132a and 134a may be formed only in the light passing holes 131a and 133a that are most adjacent to the eye (or the viewing hole 110) while the mask 100 is worn on the face.

Alternatively, the light blocking ribs 132a, 132b, 132c, 134a, 134b may be formed at the light passing holes 131a most adjacent to the eyes (or viewing hole 110) and some other light passing holes 131b, 131c, 133a, 133b adjacent to the light passing holes.

The light blocking ribs 132, 134 may be formed along an edge of the light passing holes 131, 133, and the light blocking ribs 132, 134 may be formed along a part of the edge of the light passing holes 131, 133 adjacent to the eye (or viewing hole 110).

The light blocking ribs 132, 134 may be formed to be thin with a predetermined thickness such as a shape of a rib. In this case, the thickness of the light blocking ribs 132, 134 is not limited to a specific thickness and may be any thickness capable of blocking light emitted from the LED module 200 so that the light may not be passed or lit.

The light blocking ribs 132 and 134 may protrude at a predetermined length toward the first surface of the outer case 121. In this case, the protruding length may be enough as long as the light blocking ribs 132 and 134 can block the light emitted from the LED module 200 corresponding to the light passing holes 131 and 133 from being irradiated to the eye.

For convenience, a light passing hole most adjacent to the viewing hole 110 is defined as first light passing holes 131a, 133a and a light passing hole most adjacent to the first light passing hole in a direction gradually distancing from the viewing hole 110 is defined as second light passing hole 131b, 133b, respectively.

The second light blocking ribs 132b, 134b formed on the second light passing holes 131b, 133b may have a protruding length which is enough to block the light emitted from each LED module 200 corresponding to the second light passing holes 131b, 133b from being irradiated to the eye.

The protruding length of the second light blocking ribs 132b, 134b formed on the second light passing holes 131b, 133b may be formed to be the same as a protruding length of the first light blocking ribs 132a, 134a formed on the first light passing holes 131a, 133a.

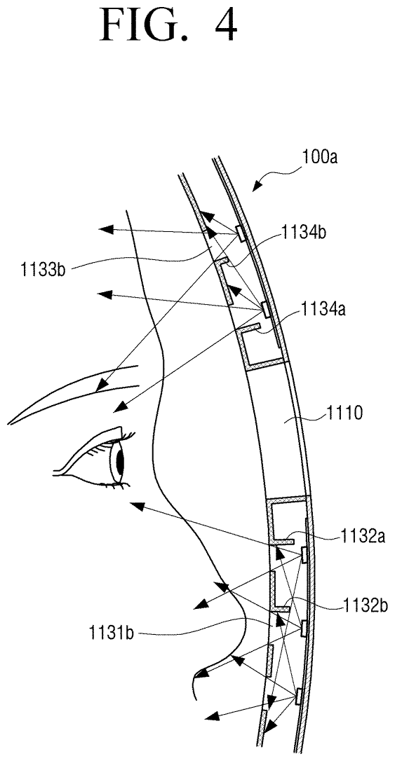

FIGS. 4 to 8 are partial sectional diagrams illustrating various arrangement examples of a light blocking rib provided in a skin care apparatus according to an embodiment.

It is also possible that the protruding lengths of the first light blocking ribs and second light blocking ribs 132a, 134a; 132b, 134b respectively formed on the first and second light passing holes 131a, 133a; 131b, 133b are formed to be different.

As an example, a mask 100a illustrated in FIG. 4 may have a gradually shortened protruding length of the light blocking rib walls 1132a, 1132b, 1134a, and 1134b positioned at a position that gradually distances from a position most adjacent to the viewing hole 1110.

In this example, a light blocking rib may not be formed in a farther position from the viewing hole 1110, for example, in the remaining light passing holes located farther from the second light passing holes 1131b and 1133b. This can be applied when light emitted to the facial skin through the remaining light passing holes is not directly irradiated with the eye.

As illustrated in FIG. 2, the light blocking ribs 132, 134 may protrude with respect to the first surface or second surface of the inner case 123 at an approximately a right angle.

The embodiment is not limited thereto and the plurality of light blocking ribs 132, 134 may be positioned to be inclined with respect to the inner case 123 at a predetermined angle.

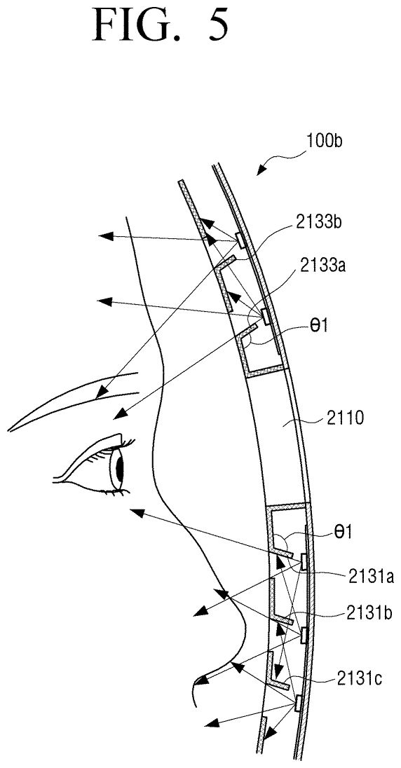

For example, a mask 100b shown in FIG. 5 may be positioned to be inclined at a predetermined angle (θ1) in a direction of distancing from a viewing hole 2110 of the plurality of light blocking ribs 2131a, 2131b, 2131c, 2133a, and 2133b. Here, the predetermined angle (θ1) may be an angle that forms an obtuse angle with the second surface of the inner case 123. In this case, the plurality of light blocking ribs 2131a, 2131b, 2131c, 2133a, and 2133b may all be inclined at the same angle.

It is not necessary that all the plurality of light blocking ribs are positioned to be inclined at the same angle, and the light blocking ribs may be positioned to be inclined at different angles.

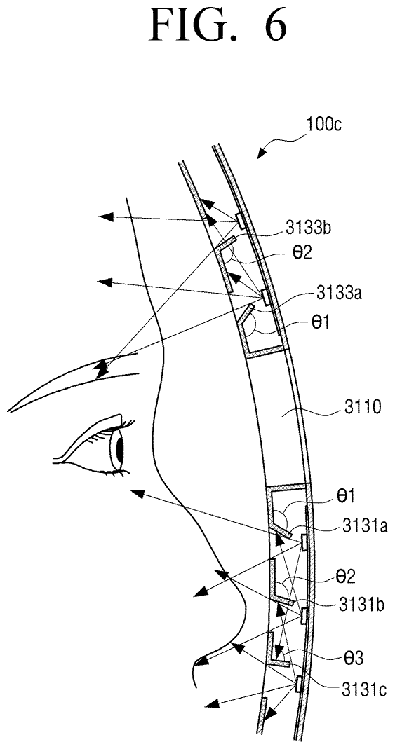

As an example, in a mask 100c shown in FIG. 6, the light blocking ribs 3131b, 3131c; 3133b positioned at a location farther away from the light blocking ribs 3131a; 3133a most adjacent to the viewing hole 3110 may be positioned such that the incline angle gets narrower (θ1>θ2>θ3).

The plurality of light blocking ribs shown in FIGS. 5 and 6 are shown to have the same protruding lengths, but the embodiments are not limited thereto, and it is also possible to form the protruding lengths of the plurality of light blocking ribs to be different from each other. For example, the protruding length of the light blocking rib that is most adjacent to the viewing hole may be formed to be longest, and the protruding length of the light blocking rib at a position gradually distancing from the viewing hole can be gradually shortened. As such, when the protruding lengths of the plurality of light blocking ribs are formed differently, the protruding length of each light blocking rib is formed by considering that the light emitted through the plurality of light passing holes is not irradiated to the eye.

It has been described that a protruding direction of the plurality of light blocking ribs is directed toward the first surface of the outer case 121, but the embodiment is not limited thereto, and it is also possible to form the plurality of light blocking ribs to protrude in a direction toward the face when the mask is worn.

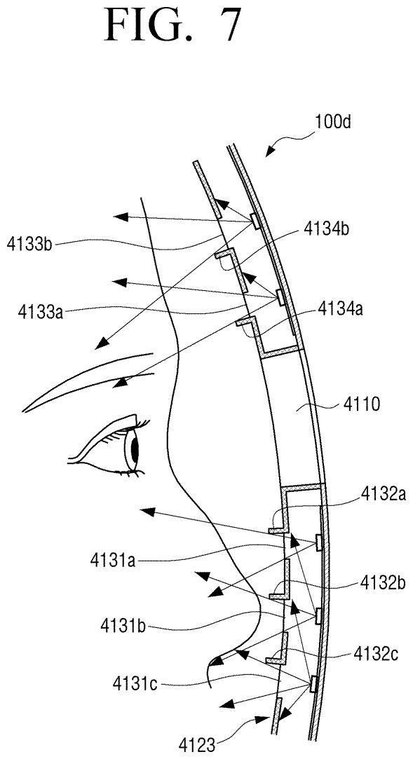

As an example, a mask 100d shown in FIG. 7 may be formed such that a plurality of light blocking ribs 4132a, 4132b, 4132c; 4134a, 4134b have a protruding length to prevent light emitted from the LED module 200 from being irradiated to the eye. In this case, the plurality of light blocking ribs 4132a, 4132b, 4132c, 4134a and 4134b are formed to have a protruding length such that the mask 100d is not in contact with the face of the user while the mask 100d is worn on the face. In FIG. 7, reference numerals 4131a, 4131b, 4131c, 4133a, and 4133b denote light passing holes, respectively.

The plurality of light blocking ribs 4132a, 4132b, 4132c; 4134a and 4134b may protrude at an approximate right angle with respect to the first surface of the inner case 4123. In this case, the plurality of light blocking ribs 4132a, 4132b, 4132c; 4134a, 4134b can be formed to have the same protruding length.

The embodiment is not limited thereto and the plurality of light blocking ribs 4132a, 4132b, 4132c; 4134a, 4134b may be formed to be different from each other. For example, the length of the light blocking ribs 4132b, 4132c; 4134b positioned at a position that gradually distances from the light blocking ribs 4132a; 4134a at a position most adjacent to a viewing hole 4110 may be formed to be gradually shorter.

In addition, it is also possible to form light blocking ribs 4132a and 4134a at a position most adjacent to the viewing holes 4110 among the plurality of light blocking ribs 4132a, 4132b, 4132c;4134a, 4134b, and omit remaining light blocking ribs 4132b, 4132c; 4134b.

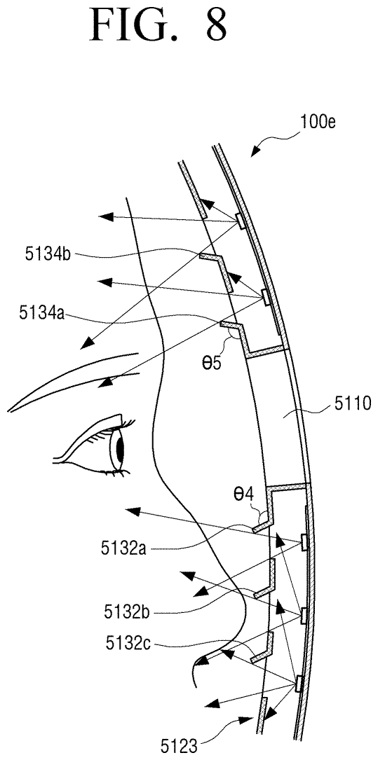

Referring to FIG. 8, a mask 100e may be positioned such that the plurality of light blocking ribs 5132a, 5132b,5132c; 5134a, 5134b are inclined at a predetermined angle θ4, 05 in a direction of distancing from the viewing hole 5110. In this case, the plurality of light blocking ribs 5132a, 5132b, 5132c; 5134a, 5134b may be positioned at an obtuse angle from the first surface of the inner case 5123. The inclined angles θ4, 05 of the plurality of light blocking ribs 5132a, 5132b, 5132c; 5134a, 5134b may be the same or different.

The plurality of light blocking ribs 5132a, 5132b, 5132c; 5134a, 5134b may be formed to have different protruding lengths. For example, the protruding length of the light blocking ribs 5132b, 5132c; 5134b which are positioned at a location that is farther away from the light blocking ribs 5132a; 5134a located at the most adjacent to the viewing hole 5110 may be formed to be gradually shorter.

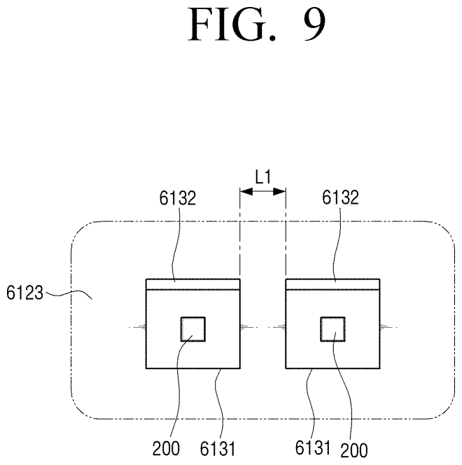

FIGS. 9 to 11 are schematic diagrams illustrating various arrangements of an LED module and a light passing hole formed on the skin care apparatus according to an embodiment.

The mask according to the above-described examples may have a plurality of light passing holes in, for example, a band shape, and a plurality of LED modules 200 may be positioned to correspond to one light passing hole. The embodiment is not limited thereto and as shown in FIG. 9, the LED module 200 may be positioned such that each LED module 200 corresponds to each of the light passing holes 6131 formed in the inner case 6123. In this case, the plurality of light passing holes 6131 may be formed to have a length shorter than that of the band-shaped light passing holes described above, and may be rectangular.

The plurality of light passing holes 6131 may be spaced apart from each other by a predetermined distance from the inner case 6123. In this case, an interval L1 between each light passing hole 6131 is considered to be evenly irradiated to the entire face excluding the eyes.

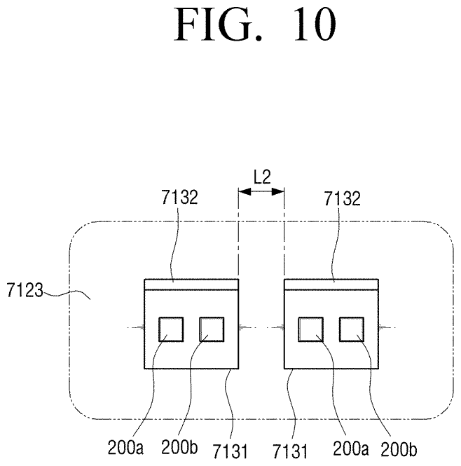

Referring to FIG. 10, two LED modules 200a and 200b may be positioned to correspond to one light passing hole 7131 formed in an inner case 7123. In this case, the amount of light emitted from one of the light passing holes 7131 may be greater than the amount of light emitted from the light passing hole 6131 of FIG. 9. Thus, an interval L2 between each light passing hole 7131 may be longer than the distance L1 between the light passing holes 6131 shown in FIG. 9.

Three or more LED modules can be positioned to correspond to one light passing hole 6131. In this case, the size of the light passing hole 6131 may be larger in proportion to the number of corresponding LED modules.

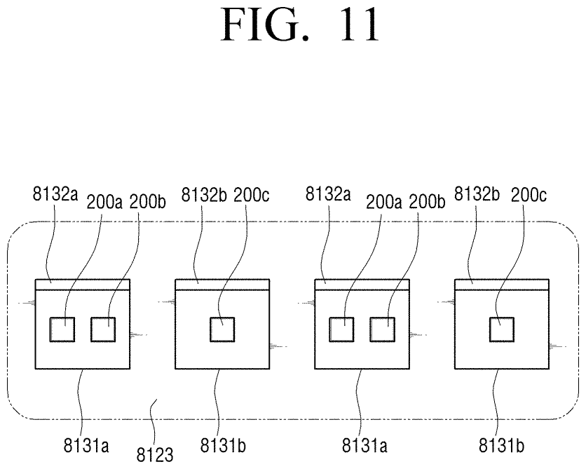

Referring to FIG. 11, the number of LED modules 200a, 200b, and 200c corresponding to each of the plurality of light passing holes 8131a and 8131b formed in an inner case 8123 can be positioned differently. For example, two LED modules 200a and 200b may be positioned in some of the light passing holes 8131a, and one LED module 200c may be positioned in the remaining light passing holes 8131b, in a corresponding manner. In this case, there may be a pattern that LED modules 200a, 200b, and 200c correspond to the respective light passing holes 8131a and 8131b, which are successively arranged at intervals, by two LED modules and one LED module in an iterative manner, respectively.

However, the arrangement pattern of the LED modules 200a, 200b, and 200c needs not be iterative as described above, but it is also possible to form an irregular arrangement pattern. As an example of an irregular arrangement pattern, each of the light passing holes corresponding to a particular portion of the face may correspond to two or more LED modules, respectively, when there is a need to irradiate more light relative to a particular portion of the face.

In each of the light passing holes 6131, 7131, 8131a, and 8131b shown in FIGS. 9 to 11, light blocking ribs 6132, 7132, 8132a, and 8132b may be formed, respectively. When each light passing holes 6131, 7131, 8131a, and 8131b is formed in a rectangular shape, edges of the light passing holes 6131, 7131, 8131a, and 8131b are four-sided. In this case, the light blocking ribs 6132, 7132, 8132a, and 8132b may be formed to protrude with a predetermined length along one side of an edge of the light passing holes 6131, 7131, 8131a, and 8131b. One side of the edge of the light passing holes 6131, 7131, 8131a, and 8131b on which the light blocking ribs 6132, 7132, 8132a, and 8132b are formed may be the side that is most adjacent to the viewing hole compared to the remaining three sides.

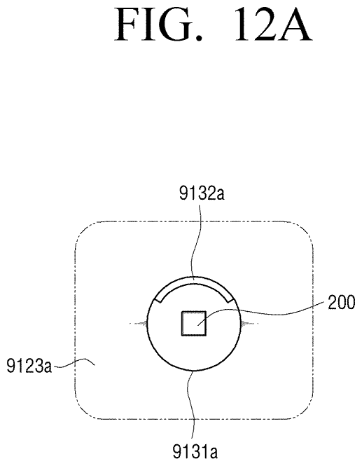

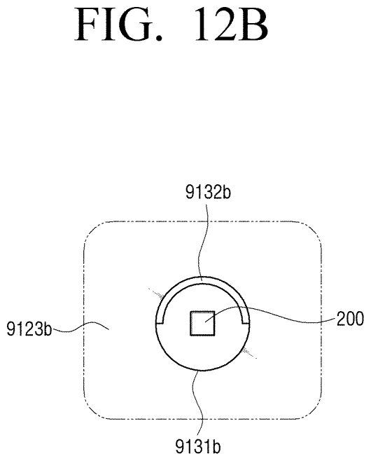

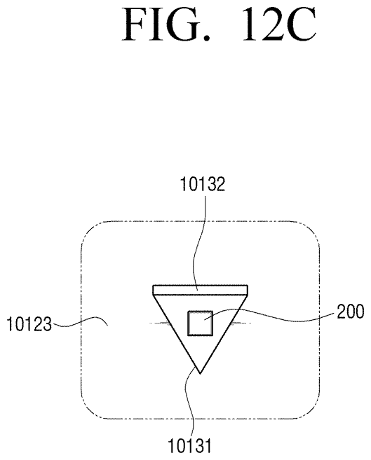

FIGS. 12A to 12C are schematic diagrams illustrating various shapes of a light passing hole formed at a skin care apparatus according to an embodiment.

Referring to FIGS. 12A to 12B, the light passing hole may have various shapes, in addition to a rectangular shape.

As an example, as shown in FIGS. 12A and 12B, the light passing holes 9131a and 9131b formed in inner cases 9123a and 9123b may be circular. In this case, the light blocking ribs 9132a and 9132b may be formed along a portion of an edge of the light passing holes 9131a and 9131b. The light blocking ribs 9132a and 9132b may be formed to have a length corresponding to an angle of approximately 90 degrees as shown in FIG. 12A along the circumference of the circular edge, or may be formed to have a length corresponding to an angle of approximately 180 degrees as shown in FIG. 12B. However, the length of the light blocking ribs 9132a and 9132b need not be limited to a length corresponding to an angle of 90 degrees and 180 degrees, and may be a length corresponding to an angle at which light can be prevented from being directly irradiated to the eye.

Referring to FIG. 12C, a light passage hole 10131 formed in inner case 10123 may be triangle. In this case, the edge of the light passing hole 10131 may be three-sided. The light blocking rib 10132 may be formed along one of three sides of the edge of the light passage hole 10131. One side of the edge of the light passing hole 10131 on which the light blocking rib 10132 is formed may be the edge that is most adjacent to the viewing hole compared to the other two sides.

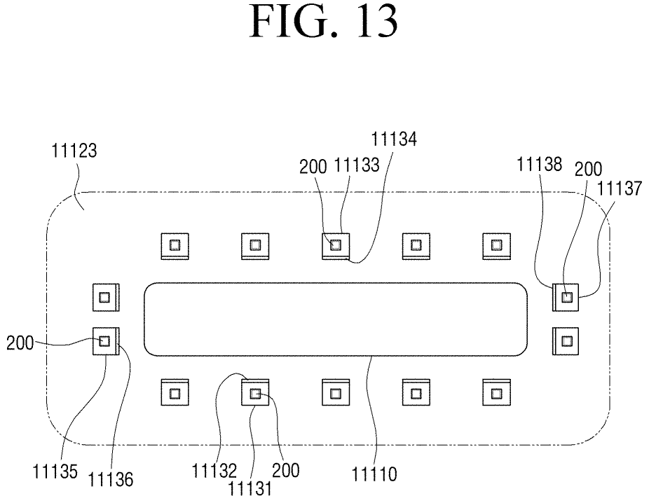

FIG. 13 illustrates a schematic diagram of an arrangement of a light blocking rib formed in each light passing hole and the light passing hole positioned around the viewing hole of the skin care apparatus according to an embodiment.

In the description above, the plurality of light passing holes formed in the mask are formed at a lower side and an upper side of the viewing hole, but the embodiment is not limited thereto, and the light passing holes may be additionally formed in a left side and a right side of the viewing hole 11110.

Referring to FIG. 13, first to fourth light passing holes 11131,11133,11135,11137 formed in an inner case 11123 may be formed along the circumference of the viewing hole 11110.

More specifically, the first and second light passing holes 11131 and 11133 may be formed at regular intervals in the transverse direction of the mask along the viewing hole 11110, respectively, on the lower side and the upper side of the viewing hole 11110. In addition, a plurality of third and fourth light passing holes 11135 and 11137 may be formed at regular intervals in the longitudinal direction of the mask along the viewing hole 11110 at the left and right sides of the viewing hole 11110.

The plurality of third and fourth light passing holes 11135 and 11137 formed at both sides of the viewing hole 11110 are spaced apart from each other at intervals, but the embodiment is not limited thereto, and may be formed in an approximate band-shape along the longitudinal direction of the mask.

As described above, the first to fourth light blocking ribs 11132, 11134, 11136, and 11138 may be formed at the edges of the first to fourth light passing holes 11131, 11133, 11135, and 11137, respectively. In this case, the first to fourth light blocking ribs 11132, 11134, 11136, and 11138 may be formed on a portion of an edge of the first to fourth light passing holes 11131, 11133, 11135, and 11137 most adjacent to the viewing hole 11110, respectively. Accordingly, the light emitted from each of the LED modules 200 corresponding to the first to fourth light passing holes 11131, 11133, 11135, and 11137 can be prevented from being irradiated to the eye.

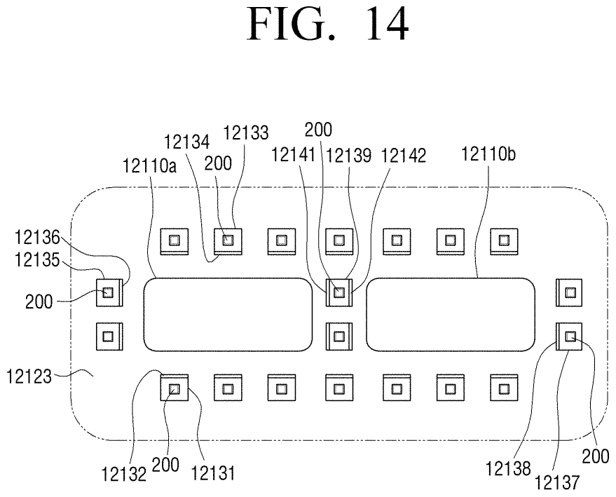

As illustrated in FIG. 14, the viewing hole formed in the mask may include first and second viewing holes 12110a, 12110b positioned at intervals to correspond to the left eye and the right eye, respectively.

In the inner case 12123, a plurality of fifth to eighth light passing holes 12131, 12133, 12135, and 12137 may be formed along the entire circumference of the first and second viewing holes 12110a and 12110b, and a plurality of ninth light passing holes 12139 may be formed between the first and second viewing holes 12110a and 12110b. At least one LED module 200 may be positioned in the fifth to ninth light passing holes 12131, 12133, 12135, 12137 and 12139, respectively.

A plurality of fifth and sixth light passing holes 12131 and 12133 may be formed at regular intervals in the transverse direction of the mask along the first and second viewing holes 12110a and 12110b, respectively, on the lower and upper sides of the first and second viewing holes 12110a and 12110b, respectively. A plurality of seventh light passing holes 12135 may be formed on the left side of the first viewing hole 12110a at a regular interval in the longitudinal direction of the mask along the viewing hole 12110a, and a plurality of eighth light passing holes 12137 on the right side of the second viewing hole 12110b may be spaced apart from each other in the longitudinal direction of the mask along the second viewing hole 12110b.

A plurality of ninth light passing holes 12139 may be formed between first and second viewing holes 12110a, 12110b along the first and second viewing holes 12110a, 12110b in a longitudinal direction of the mask at regular intervals.

One of light blocking ribs 12132, 12134, 12136 and 12138 may be formed in the fifth to eighth light passing holes 12131, 12133, 12135, and 12137, respectively. However, in the case of the plurality of ninth light passing holes 12139, two light blocking ribs 12141 and 12142 may be formed in each of the plurality of ninth light passing holes 12139. One light blocking rib 12141 of one of the two light blocking ribs 12141 and 12142 may be formed on a portion of an edge of the ninth light passing hole 12139 adjacent to the first viewing hole 12110a, and the remaining light blocking rib 12142 may be formed on a portion of the other edge of the ninth light passing hole 12139 adjacent to the second viewing hole 12110b.

Therefore, two light blocking ribs 12141, 12142 may block the light passing through the ninth light passing hole 12139 and being emitted from being irradiated toward the left eye and right eye, respectively.

The plurality of light passing holes 12135 formed on the left side of the first viewing hole 12110a, the plurality of light passing holes 12137 formed on the right side of the second viewing hole 12110b, and the plurality of light passing holes 12139 formed between the first and second viewing holes 12110a and 12110b are formed to be spaced apart from each other, but may be formed in one band shape along the longitudinal direction of the mask.

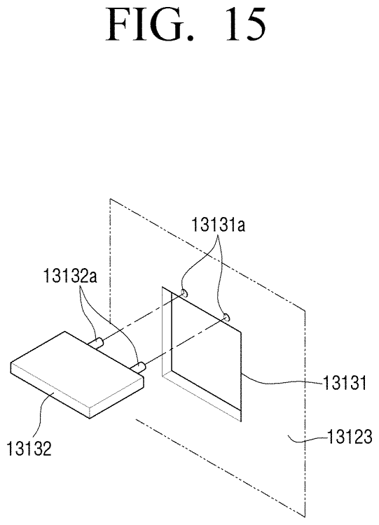

FIG. 15 illustrates an exploded perspective view of a light blocking rib provided in the skin care apparatus being formed separately from an inner case according to an embodiment.

In the disclosure, although a plurality of light blocking ribs formed in an inner case of a mask are formed integrally with the inner case, but the embodiment is not limited thereto, and a plurality of light blocking ribs may be coupled to the inner case. For example, as shown in FIG. 15, a light blocking rib 13132 formed separately from the inner case 13123 may be formed around the light passing hole 13131 formed in the inner case 13123. In this case, the light blocking rib 13132 may include a coupling protrusion 13132a that is detachably fitted into an insertion groove 13131a formed around the edge of the light passing hole 13131.

An insertion groove 13131a is arranged adjacent to the edge portion of the light passing hole 13131 on the side adjacent to the viewing hole, irradiation of the eye with light emitted through the light passing hole 13131 can be prevented by the light blocking rib 13132.

The insertion groove 13131a and a coupling protrusion 13132a can be applied with a conventional snap-fit structure, thereby enabling a stable coupling.

The light blocking rib 13132 is illustrated to be formed flat, but the embodiment is not limited thereto, and the and the light blocking rib 13132 may be formed to have a curvature according to a shape of the edge of the light passing hole.

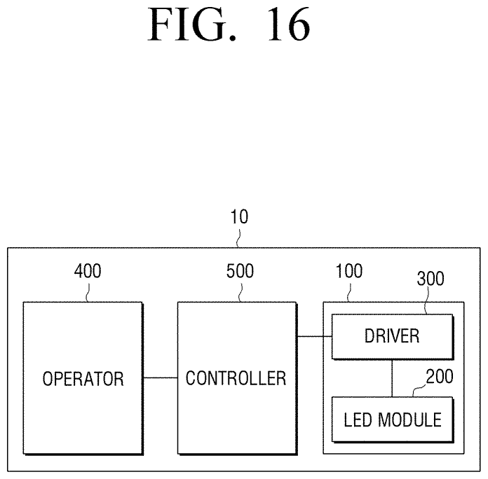

FIG. 16 illustrates a block diagram illustrating a skin care apparatus according to an embodiment.

Referring to FIG. 16, the skin care apparatus 10 according to an embodiment may include a driver 300 for turning on/off the LED module 200, an operator 400 for selecting the driving of the LED module 200, and a controller 500 for controlling the driver 300 according to an operation signal of the operator 400.

The controller 500 can be formed separately from the mask 100 and can be electrically connected to the driver 300 provided on the mask 100 through wire. Accordingly, the controller 500 can control the driver 300 by wire.

Although not shown in the drawings, when the skin care apparatus 10 includes a wireless communication module according to an embodiment, the controller 500 can wirelessly control the driver 300 as the skin care apparatus 10 is wirelessly connected to the driver 300.

The operator 400 may control the on/off of the LED module 200, and can be implemented as a key pad for user input so as to control the amount of light. The operator 400 may be provided with the controller 500 for convenience of use.

The controller 500 need not be formed separately from the mask 100 as described above, and may be positioned on a portion of the mask 100. For example, the controller 500 may be positioned at a position not exposed between the inner and outer cases of the mask.

When the controller 500 is provided in the mask 100, the operator 400 may be positioned in a portion of the mask 100. In this case, the operator 400 may be formed on a portion of the front surface or a portion of the side surface of the mask 100 that is easy to access by a user.

The skin care apparatus according to an embodiment has a viewing hole formed in a mask to ensure a sight when a user wears a mask, but the embodiment is not limited thereto, and a mask having no viewing hole may be applied.

As described above, when a mask without a viewing hole is worn and a light is irradiated on the eye, the user may close the eye. However, when the light emitted from the LED module is directly irradiated toward the eyelid even when the eye is closed, some degree of glare can be generated. The skin care apparatus according to the disclosure may prevent light from being irradiated toward an eye or an eyelid by a plurality of light blocking ribs formed inside a plurality of masks, thereby minimizing glare.

Although the present disclosure has been described with various embodiments, various changes and modifications may be suggested to one skilled in the art. It is intended that the present disclosure encompass such changes and modifications as fall within the scope of the appended claims.

What is claimed is:

1. A skin care apparatus comprising:
a plurality of light sources;
a mask comprising the plurality of light sources and a plurality of light passing holes through which light emitted from the plurality of light sources passes; and
a light blocking rib formed in at least one light passing hole among the plurality of light passing holes.

2. The skin care apparatus of claim 1, wherein the light blocking rib is formed in the at least one light passing hole that is closest to an eye of a person wearing the mask to block light irradiated in a direction of the eye of the person wearing the mask.

3. The skin care apparatus of claim 2, wherein the light blocking rib protrudes in a first direction toward an outer side of the mask, or protrudes in a second direction that is opposite to the first direction.

4. The skin care apparatus of claim 1, wherein the mask further comprises a viewing hole formed to expose an eye when the mask is worn on a face.

5. The skin care apparatus of claim 4, wherein the light blocking rib is formed in the at least one light passing hole positioned closest to the viewing hole.

6. The skin care apparatus of claim 4, wherein the light blocking rib is one of a plurality of light blocking ribs, the plurality of the light blocking ribs being formed in each of a predetermined number of light passing holes in a direction distancing from a light passing hole positioned closest to the viewing hole.

7. The skin care apparatus of claim 6, wherein the plurality of the light blocking ribs is positioned at a right angle with respect to an inner surface of the mask.

8. The skin care apparatus of claim 6, wherein the plurality of the light blocking ribs is positioned to be inclined with respect to an inner surface of the mask.

9. The skin care apparatus of claim 8, wherein the plurality of the light blocking ribs is positioned such that an angle with respect to the inner surface of the mask increases or decreases as the light blocking ribs gradually distance from the viewing hole.

10. The skin care apparatus of claim 8, wherein the plurality of the light blocking ribs is positioned at different angles with respect to the inner surface of the mask.

11. The skin care apparatus of claim 6, wherein the plurality of the light blocking ribs is formed to have a shorter length as the light blocking ribs gradually distance from the viewing hole.

12. The skin care apparatus of claim 4, wherein:
each of the plurality of light blocking holes is positioned along a length direction of the viewing hole and is positioned at intervals in a direction distancing from the viewing hole, and
the plurality of light sources is positioned at each of the light passing holes.

13. The skin care apparatus of claim 4, wherein at least one of the plurality of light sources is positioned in each of the plurality of light passing holes.

14. The skin care apparatus of claim 4, wherein the viewing hole is one of a pair of viewing holes, each of the pair of viewing holes corresponding to an eye.

15. The skin care apparatus of claim 14, further comprising:
at least one additional light passing hole positioned between the pair of viewing holes,
wherein a pair of light blocking ribs are formed in the at least one additional light passing hole, the pair of light blocking ribs positioned adjacent to the pair of viewing holes.

16. The skin care apparatus of claim 14, further comprising:
at least one additional light passing hole positioned at opposite ends of the pair of viewing holes,
wherein an additional light blocking rib is formed in each of the at least one additional light passing hole, the additional light blocking rib positioned at a side adjacent to one of the pair of viewing holes.

17. The skin care apparatus of claim 4, further comprising:
at least one additional light passing hole positioned at opposite ends that of the viewing hole,
wherein a light blocking rib is formed in each of the at least one additional light passing hole, the light blocking rib positioned in a side adjacent to the viewing hole.

18. The skin care apparatus of claim 4, wherein:
the at least one light passing hole is formed to be a circular shape, an oval shape, or a polygonal shape, and
the light blocking rib is disposed at a side of the at least one light passing hole adjacent to the viewing hole.

19. A skin care apparatus comprising:
a mask including an outer case, an inner case coupled with the outer case, and a viewing hole formed to penetrate the outer case and the inner case;
a plurality of light sources positioned in the outer case;
a plurality of light passing holes formed in the inner case through which light emitted from the plurality of light sources passes; and
a plurality of light blocking ribs, the plurality of light blocking ribs positioned at edges of each of the plurality of light passing holes, and the plurality of light blocking ribs protruding in a first direction toward the outer case or protruding in a second direction opposite to the first direction.

20. The skin case apparatus of claim 19, wherein:
the plurality of light blocking ribs are formed to be right-angled or inclined with respect to the inner case, respectively, and
the inner case is made of an opaque material.
